(19) United States
(12) Patent Application Publication
(10) Pub. No.: US 2007/0241670 A1
(43) Pub. Date: Oct. 18, 2007

(54) Organic materials with phosphine sulfide moieties having tunable electric and electroluminescent properties

(75) Inventors: Linda Susan Sapochak, Kennewick, WA (US); Paul Edward Burrows, Kennewick, WA (US); Asanga Bimalchandra, Richland, WA (US)

Correspondence Address: Douglas E. McKinley, Jr., P.O. Box 202, Richland, WA, 99352, US

(73) Assignee: Battelle Memorial Institute

(21) Appl. No.: 11/406,143

(22) Filed: Apr. 17, 2006

(51) Int. Cl. H01J 1/62; C07F 9/06; C07F 9/6553; C07F 9/58; C07D 487/22

(52) U.S. Cl. 313/504; 540/145; 540/128; 546/22; 548/412; 549/6; 544/214; 548/155

(57) ABSTRACT

A material comprising one or more phosphine sulfide moieties, the phosphorus atom of each of said phosphine moieties bonded by single bonds to at least two outer groups. The material is substantially purified and configured as part of a circuit. The material is preferably configured as an organic light emitting device having an anode layer, a cathode layer, and at least one organic layer interposed between the anode and cathode layer, wherein at least one of said organic layers comprises the substantially purified material having one or more phosphine sulfide moieties, and wherein the phosphorus atoms of each of said phosphine sulfide moieties is further bonded by single bonds to two outer groups.

The invention was made with Government support under Contract DE-AC0676RLO 1830, awarded by the U.S. Department of Energy. The Government has certain rights in the invention.

### TECHNICAL FIELD

This invention relates to materials with charge transporting and electroluminescent properties. More specifically, this invention relates to phosphine sulfides in organic molecules and the use of those molecules in electroluminescent devices.

### BACKGROUND OF THE INVENTION

In U.S. application Ser. No. 11/035,379 filed Jan. 12, 2005 and titled ORGANIC MATERIALS WITH TUNABLE ELECTRIC AND ELECTROLUMINESCENT PROPERTIES, by Linda S. Sapochak, et al., certain types of electroluminescent phosphine oxide materials were disclosed, together with their use in various devices. (This and all other patents, papers or other publications referenced herein are hereby incorporated in their entirety by this reference). While the advances enabled by the phosphine oxide materials disclosed in this prior application are significant, there exists a continued need in the art for improved materials. For example, and not meant to be limiting, improved materials might exhibit higher electrical efficiency, more favorable operating characteristics, and/or improved physical characteristics. The present invention addresses this ongoing need.

### SUMMARY OF THE INVENTION

Accordingly, it is a general object of the present invention to provide a new class of materials for use in electric and electroluminescent devices. These materials are generally described as organic materials with one or more phosphine sulfide moieties. Generally, it is preferred that two or more phosphine sulfide moieties are utilized, and that these phosphine sulfide moieties are joined by a bridging group. Each of the phosphine sulfide moeities is further bonded by single bonds to at least two outer groups. The outer groups may be linked or bonded to one and another, thereby approximating a single group or a ring, however for purposes of this disclosure they are still referred to as two outer groups, since they are bonded to the phosphorus atom via two single bonds. Outer groups, as the term is used herein, are bound to a single phosphine sulfide moiety. Bridging groups, as the term is used herein, are bound to two or more phosphine sulfide moieties. The entire molecule; the one or more phosphine sulfide moieties, the bridging group, and the two outer groups (whether bonded together or not bonded), is hereinafter referred to as a “phosphine sulfide.” Examples of the general structure of the present invention are shown in FIGS. 1 and 2. A single phosphine sulfide moiety is shown in FIG. 1, and examples of a di-bonded and a tri-bonded phosphine sulfide moiety are shown in FIG. 2. As shown in FIG. 2, the phosphine sulfide structures of the present invention can generally be used in oligomer and polymer structures as indicated by the subscript “n” in FIG. 2. As will be recognized by those having skill in the art, this definition would therefore include diphosphine sulfide, triphosphine sulfide, and other polyphosphine sulfides. Further, the bridging groups themselves may contain phosphine sulfides.

The phosphine sulfides of the present invention are further purified and configured as part of a circuit. As used herein, the phrase “configured as part of a circuit” means that the phosphine sulfides are configured to be exposed to an external stimulus, including but not limited to an electrical current, a voltage, a light source, or a temperature gradient. When the materials are exposed to an external stimulus, a predictable response is elicited. Thus, the present invention is a new class of materials, which, in part, are defined by their electrical and electroluminescent properties, and these properties are thus a fundamental aspect of the invention. Preferred embodiments of the present invention include circuits utilizing the materials of the present invention as an OLED, a photodetector, a solar cell, a thin film transistor, a bipolar transistor, a sensor, and wherein the circuit is incorporated in an array to form an information display. For example, in an OLED, the novel materials could potentially function as an emitting layer, as a phosphorescent dopant, an electron transporting layer, a hole blocking layer, an exciton blocking layer, a host layer which transfers energy to a light emitting dopant, or a combination of any of the above.

One important difference between the phosphine sulfides of the present invention and the phosphine oxides previously reported is the main mechanism of light emission in the solid state from the phosphine sulfides is phosphorescence and the main mechanism of light emission in the solid state from the phosphine oxides is fluorescence. This difference is particularly pronounced at room temperatures, where phosphine oxides exhibit essentially no phosphorescence and phosphine sulfides exhibit observable phosphorescence. This characteristic is readily exploited in the use of phosphine sulfides in devices that are normally operated at or near room temperatures.

In a transistor, either bipolar or thin film, the material would function as the charge transporting active semiconductor layer in a similar manner to doped silicon in a conventional field effect transistor. In a solar cell, the material would function as a charge transporting or exciton blocking layer.

As stated above, it is a critical aspect of the present invention that the materials be purified. Only phosphine sulfides that are substantially purified will exhibit the electrical and electroluminescent properties which define the materials of the present invention. While not meant to be limiting, some stages of the purification process are generally performed when the materials are synthesized. A variety of techniques are known that produce phosphines which are typically used as precursors of the present invention. Either when formed, or when utilized in an application, it is typical that one or both of the phosphine groups formed by these methods will eventually be oxidized, thereby producing a mixture of the phosphine sulfide and unreacted phosphine. To purify this mixture, successive sublimation is preferred. “Successive sublimation” simply means sublimating the various species one at a time under vacuum, taking advantage of the fact that typically the phosphine sulfide species will have much different sublimation temperatures than the unreacted phosphine species, even though the bridging groups and outer groups may be the same. The sublimed species also have different physical appearances, further simplifying the process.

Accordingly, the reasons successive sublimation is preferred are fairly straightforward. It is effective at producing the required degree of purification, it generally requires no additional solvents or other materials be introduced into the process, and it generally generates a minimum amount of waste. However, while sublimating each of phosphine sulfide and phosphine species is an effective method for producing phosphine sulfide materials of acceptable purity, any method that produces substantially the same result; a substantially purified phosphine sulfide, should be understood as being encompassed by the present invention. Further, it should be understood that the successive sublimation that produces the diphosphine sulfide species of the present invention must be performed much more carefully and slowly than is typical. Rapid heating and/or poor vacuum in the sublimation process will not produce the purity required for the present invention, even though the substance may appear to be pure using standard chemical characterization techniques, such as thin layer chromatography, high pressure liquid chromatography, NMR, and elemental analysis. Accordingly, as used herein, it should be understood that a phosphine sulfide has been “substantially purified” when it will no longer produce any phosphine structures that are not fully oxidized at the phosphine moiety that are detectable by NMR when the mixture has been heated to a temperature above the sublimation temperature of the non-oxidized phosphine structures, but below the sublimation temperature of the phosphine sulfide at a vacuum of at least 10⁻⁶ Torr and for a period of at least 24 hours. As will be recognized by those having ordinary skill in the art, the process of producing the “substantially pure” phosphine sulfides of the present invention will typically remove many other undesirable impurities, and other chemical techniques can and should be used to remove such impurities. However, for purposes of defining “substantially pure,” these other impurities should not be viewed as limiting the scope of the present invention. Further, while successive sublimation is typically required to produce the requisite purity, it may not be used at all, or it may be used in conjunction with other standard chemical separation procedures such as column chromatography. The inventors have determined that successive sublimation is an efficient and effective separation regime to produce materials of the requisite purity.

As will be recognized by those having ordinary skill in the art, certain polymeric and large oligomeric molecules are not amenable to vacuum sublimation but are still useful as a thin film circuit element when applied by solution-based coating techniques such as spin-coating or printing. The purification requirements for such materials is generally similar to those described above, with the exception that purification is performed on the precursor monomer or oligomer before assembly of the final phosphine sulfide.

One of the principle advantages of the present invention is that by selecting appropriate bridging and outer groups, the present invention enables designers to “tune” the electrical and electroluminescent characteristics of the materials. Generally, aromatic, heteroaromatic, alicyclic and aliphatic compounds may be used for the bridging group and for the outer groups. The bridging group can also include one or more phosphine sulfide moieties, each bonded to an organic molecule. The particular selection of each will determine the electrical and luminescent properties of a specific material. Accordingly, the materials may be viewed as “tunable” meaning that a material with particular photophysical properties (such as triplet exciton energy) may be synthesized for use in a particular application which requires that property. This is a result of the fact that the phosphine sulfide moiety restricts electron conjugation between the bridging and outer groups, and between the outer groups themselves. The fact that the bridging and outer groups are isolated from each other, allows the photophysical properties of the bridging and outer groups to be maintained in the molecule.

In theory, there are several choices of functionalities that could be used as a point of saturation, (or to restrict conjugation) including sp³-C, Si, P═O, and P═S. Although each functionality can provide materials with tunable photophysical and material properties, only P═O and P═S moieties can aid electron or charge transport. The reason lies in the fact that the π-electron cloud is drawn towards the P═X moiety, making the attached aryl groups electron deficient. In contrast, sp³-C or Si have weak donating inductive effects. The present invention is derived from the surprising discovery that in addition to the fact that the π-electron cloud is drawn towards the P═X moiety in P═S compounds, these P═S compounds have unique photophysical properties that distinguish them from the P═O compounds. Specifically, as described above, the phosphine sulfides cause the attached aryl groups to exhibit observable phosphorescence at room temperatures in the solid state. The combination of the capability to phosphoresce without the presence of a heavy metal and without cooling to liquid nitrogen temperatures is only present in a limited number of organic molecules. The present invention provides a heretofore unattained capability when compared with these other molecules; specifically the present invention provides phosphorescence materials that may be tuned.

The lowest energy component (bridging group or outer group) will define the triplet state and highest occupied molecular orbital energies for the entire molecule. Accordingly, a specific requirement for a material may be met by choosing the appropriate bridging and outer groups, without having to consider the electrical interaction between the two. The present invention is therefore the entire class of materials having the phosphine sulfide moiety, as the disclosure of this moiety has enabled a broad range of materials to be tuned to a wide variety of specific applications.

For example, materials such as naphthalene or biphenyl whose wide bandgap and high triplet state energies are desirable, but whose physical properties are unsuitable for practical device applications, can be combined and incorporated into the materials of the present invention, preserving their desirable photophysical properties (wide bandgap and high triplet state energies) while making them physically amenable to practical device applications, including but not limited to, thin film formation.

While not meant to be limiting, the use of the materials of the present invention as charge transporting host materials in organometallic phosphor-doped electroluminescent devices provides an excellent example of how the phosphine sulfide materials may be “tuned” for a specific application. For example, a material suitable as a charge transporting host for a blue phosphorescent OLED is achieved by selecting the bridging group as dibenzothiophene and all outer groups as phenyl to give 2,8-bis(diphenylphosphine sulfide) dibenzothiophene (shown as PS15 in FIG. 3). The bridging group is thus the lowest energy group attached to the phosphine sulfide moieties, and the triplet state energy of PS15 is almost identical to dibenzothiophene (E_{T}˜3.0 eV). Thus, the melting point of the overall molecule is much higher than the dibenzothiophene, while the triplet energy of the dibenzothiophene is preserved.

Materials suitable as charge transporting hosts for green phosphorescent OLEDs can be achieved, for example, by selecting the bridging group as biphenyl and all outer groups as phenyl to give 4,4′-bis(diphenylphosphine sulfide) biphenyl (shown as PS1 in FIG. 3). For PS1, the bridging group is the lowest energy group attached to the phosphine sulfide moieties, and the triplet state energy of PO1 is almost identical to biphenyl (E_{T}=2.8 eV). If the phosphine sulfide is engineered by selecting the bridging group as biphenyl and the outer groups are selected as phenyl and 1-naphthyl to give 4,4′-bis(1-naphthylphenylphosphine sulfide) biphenyl. The 1-naphthyl is the lowest energy group attached to the phosphine sulfide moieties, and the triplet state energy of this phosphine sulfide would be characteristic of naphthalene (E_{T}=2.6 eV). As with the blue OLED, tuning the materials in this manner achieves a material exhibiting similar photophysical properties to naphthalene, but with a much higher melting point. Suitable outer groups include, but are not limited to, aryl, heteroaryl, cycloalkyl, or alkyl groups, as well as, R-substituted derivatives of these groups, where the substituted derivative is an alkyl, aryl, heteroaryl, halo, amino, hydroxyl, alkoxy, cyano, halogenated alkyl, aryl or heteroaryl. Preferred outer groups are shown in FIG. 4 wherein x denotes a repeating unit, and can be an integer between 1 and 6. These outer groups can be used alone or in combinations to form the phosphine sulfide structures shown FIGS. 1 and 2.

Suitable bridging groups therefore include, but are not limited to, aryl, heteroaryl, cycloalkyl, or alkyl groups. Preferred bridging groups include, but are not limited to, difunctional or multifunctional groups (i.e., substituted at two or more positions) and selected from benzene, naphthalene, pyrene, stilbene, diphenylethyne, pyridine, quinoline, thiophene, phenylene vinylene, thienylene vinylene, biphenyl, diphenylmethane, bithiophene, bipyridine, porphyrins and metalloporphyrins, phthalocyanines and metallophthalocyanines, perylene or naphthalene, carbazole, dibenzothiophene, dibenzofuran, 2,4-diphenyl-1,3,5-triazine, 2,4,6-triphenyl-1,3,5-triazine, 2,4,6-triphenylpyridine, diphenylmethane, benzophenone, dibenzosulfoxide, and substituted versions with R as defined above. Specific examples are shown in FIG. 5 wherein x denotes a repeating unit, and can be an integer between 1 and 10.

### BRIEF DESCRIPTION OF THE DRAWINGS

FIG. 1 is a schematic drawing of a general structure of the mono phosphine sulfide embodiment of the present invention.

FIG. 2 is a schematic drawing of the general structure of the A. di phosphine sulfide and B. tri-or poly (n)-phosphine sulfide embodiment of the present invention.

FIG. 3 shows examples of structures tuned to be used as the conductive host in blue and green organometallic phosphor doped OLED in accordance with the present invention.

FIG. 4 shows the structures of preferred outer groups utilized in the present invention.

FIG. 5 shows the structures of preferred bridging groups utilized in the present invention.

FIG. 6 shows the normalized absorption, and emission intensities as a function of wavelength for a preferred embodiment of the present invention (4,4′-bis(diphenylphosphine sulfide)biphenyl) (PS1) compared to the bridging group, biphenyl.

FIG. 7 is the emission spectra of PS1 showing phosphorescence from the solid state film at room temperature, compared with the emission observed in a frozen solution at 77 K. Spectrum after detector delay of 0.001 s is also shown confirming emission is phosphorescence (lifetime˜0.5 s).

FIG. 8 shows the electroluminescence spectrum for a preferred embodiment of the present invention composed of: ITO/200 Å CuPc/400 Å PS1/6 Å LiF/1000 ÅAl. The spectrum is compared to the photoluminescence (PL) spectrum obtained when a PS1 film is excited with photons.

FIG. 9 is a graph of current density verses voltage from a preferred embodiment of the present invention composed of: ITO/200 Å CuPc/400 Å PS1/6 Å LiF/1000 Å Al.

FIG. 10 is the general device structure used to test PS1 as a phosphorescent dopant in an organic light emitting device.

FIG. 11 is the general device structure used to test PS1 as a host material for a blue/green organometallic phosphor in an organic light emitting device.

FIG. 12 is a graph of external quantum efficiency verses drive current density from a preferred embodiment of the present invention composed of: ITO/200 Å CuPc/200 Å NPD/50 Å TCTA/200 Å 20% Firpic: PS1/200 Å PS1/6 Å LiF/1000 Å Al. Inset 1: current density verses voltage for same device.

FIG. 13 are the orbital amplitude plots of the HOMO and LUMO of N1 and PO1 compared to the energy degenerate set of the non-bonding electrons on sulfur (HOMO, HOMO-1, HOMO-2, HOMO-3), HOMO-4, and LUMO of PS1.

FIG. 14 is a comparison of the change in computed HOMO and LUMO energies (B3LYP/6-31G*) caused by 4,4′-disubstitution of the bridging group, biphenyl (Bp) with diphenylamino (N1), diphenylphosphine oxide (PO1), and. diphenylphosphine sulfide (PS1), also showing the non-bonding electrons on sulfur.

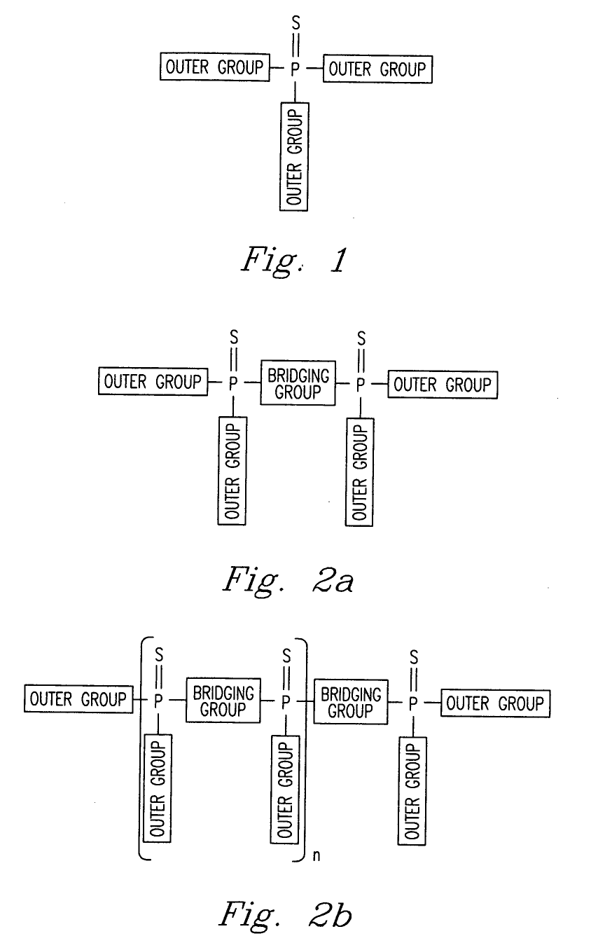

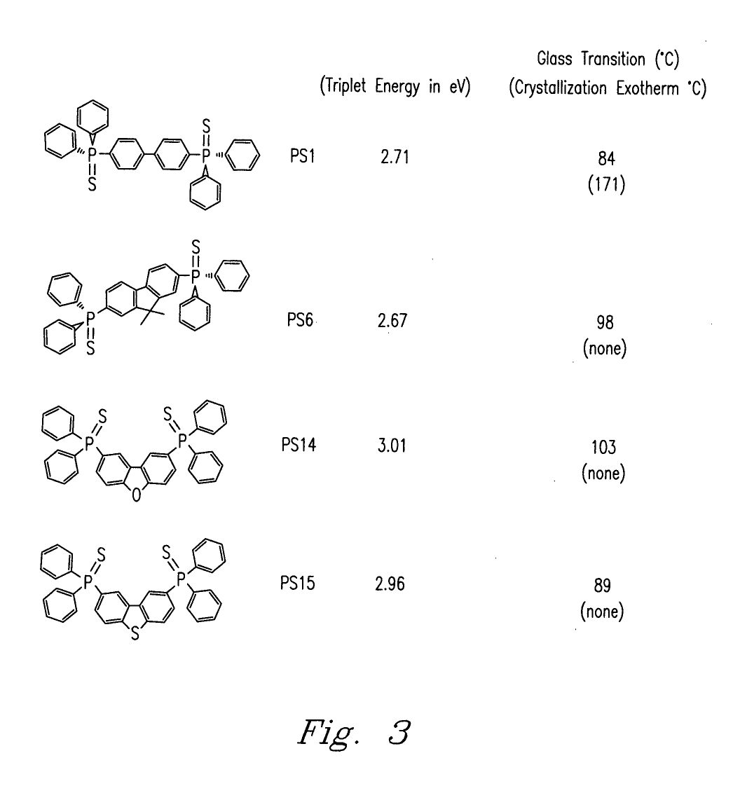

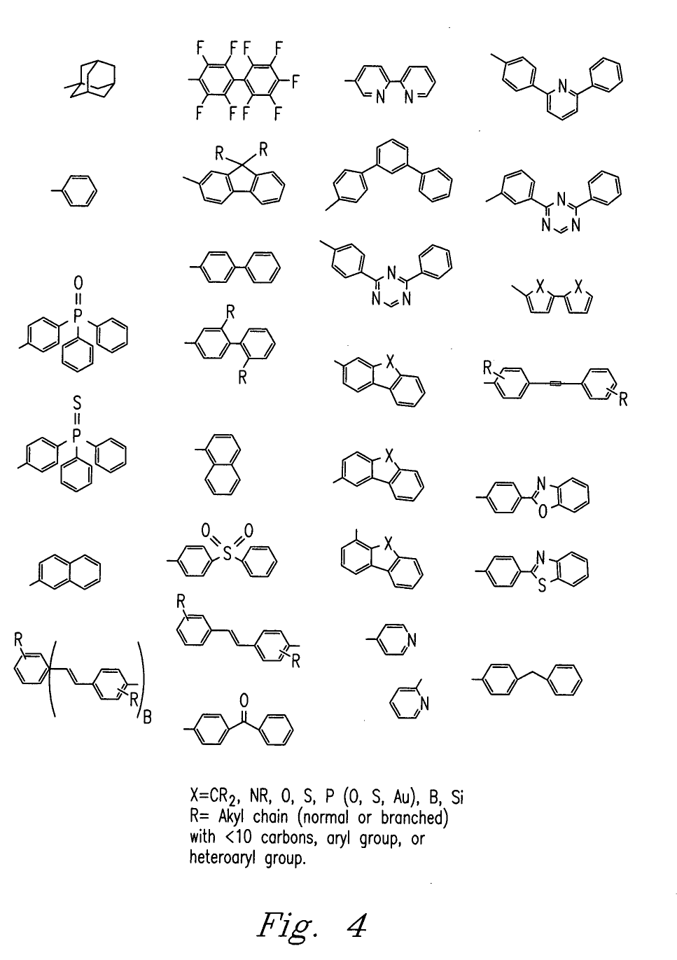

### DETAILED DESCRIPTION OF THE PREFERRED EMBODIMENTS

A series of experiments were conducted to demonstrate a preferred embodiment of the present invention, and its successful use as the active component of an exemplary electronic device. 4,4′-bis(diphenylphosphine sulfide) biphenyl (hereafter PS1) was used as an example of the present invention to demonstrate how the phosphine sulfide moieties of the present invention restrict electron conjugation and provide a wide optical gap, electron transporting material that emits blue phosphorescence in the solid state.

PS1 was obtained by oxidation of 4,4′-bis(diphenylphosphine)biphenyl (P1). The synthesis was performed as follows. All chemicals were obtained from Aldrich Chemical Co. and used as received unless noted otherwise. All glassware was thoroughly dried prior to use. 4,4′-bis(diphenylphosphine)biphenyl (P1) [CAS # 4129-44-6] was formed by providing a 250 mL, 3-neck round bottom flask equipped with a stir bar and thermometer filled with argon. The flask was charged with 3.21 g [0.01 moles] of 4,4′-dibromobiphenyl and 90 mL of anhydrous THF. Once all the 4,4′-dibromobiphenyl had dissolved the mixture was cooled to −66° C. n-Butyl lithium [0.02 moles] was added dropwise using a syringe. Once the addition was completed, stirring was continued another hour at −66° C. after which the reaction mixture was allowed to warm up and stabilize at 0° C. for a 3-hour period. The reaction flask was cooled again to −66° C. prior to addition of 3.58 ml chlorodiphenylphosphine [0.02 moles] by syringe. As the addition was completed the color of the reaction mixture became pale yellow. The mixture was allowed to stir for 3 hours at −66° C. before gradual warming to room temperature overnight. The reaction was then quenched with 2 mL of degassed methanol and all volatiles removed under reduced pressure. The crude white solid obtained was dissolved in degassed CH₂Cl₂ and immediately filtered through a short column of Celite (under nitrogen atmosphere). The CH₂Cl₂ was removed and the white solid was digested in degassed ethanol and gravity filtered affording 4.70 g of crude P1. A silica column was used with CH₂Cl₂ as the solvent to separate the P1 (R_{f}−0.99) from its monoxide (R_{f}−0.03). Removal of volatile solvents under vacuum resulted in 4.16 g of chemically pure P1 (80%).

The resultant material was characterized as follows. NMR spectra were obtained using a Varian Infinity CMX 300-MHz NMR spectrometer at the following frequencies: 300 MHz (¹H), 121.4 MHz (³¹P) 100.6 MHz (¹³C). Tetramethylsilane (TMS) was used as an internal reference for ¹H and ¹³C spectra and the ³¹P signals were externally referenced to 85% H₃PO₄. Elemental analysis was performed by Desert Analytics Laboratories, Tucson, Ariz. USA. Melting points of pure materials were determined by differential scanning calorimetry (DSC) using a Netzsch simultaneous thermal analyzer (STA400) with a heating rate of 10° C./min under N2 gas. Indium metal was used as the temperature standard. Elemental analysis was performed by Desert Analytics Laboratories, Tucson, Ariz. USA. The findings, and comparisons with literature values, were as follows: Mp: 195° C. (DSC) (mp 192.5° C.-194° C.). Anal. calc. for C₃₆H₂₈P₂: C, 82.74; H, 5.40; found: C, 82.73; H, 5.42. ¹H NMR (CDCl₃, 295 K): δ 7.56 (m, 4H), 7.3-7.4 (24H). ¹³C{¹H} NMR (CDCl₃, 295 K): δ 6140.74 (s, 1/1′, 2C), 137.30 (d, ¹J_{PC}=12 Hz, ipso-Ph, 4C), 136.1 (d, 4/4′, ¹J_{PC}=12 Hz 2C), 134.19 (d, ²J_{PC}=18 Hz, 3/3′, 4C), 133.78 (d, ²J_{PC}=18 Hz, o-Ph, 8C), 128.8 (s, p-Ph, 4C), 128.56 (d, ³J_{PC}=7 Hz, m-Ph, 8C), 127.06 (d, ³J=7 Hz, 2/2′, 4C). ³¹P NMR (CDCl₃, 295 K): δ 5.62.

4,4′-bis(diphenylphosphine sulfide)biphenyl (PS1) was synthesized as follows. A 500 mL round bottomed flask was charged with 3.0 g of P1 [0.0057 mol], 30 mL of CH₂Cl₂, and 0.384 g (0.012 mol, 2.1 eq) of sulfur. After stirring the reaction mixture overnight, the organic solvents were evaporated under vacuum to give an off-white solid. Unreacted P1 and other impurities were removed by digestion in methanol to afford 2.75 g (82%) of chemically pure PS1 as evident by TLC (SiO₂: CH₂Cl₂). Mp. 305° C. (DSC, 10 K/min). Anal. calc. for C₃₆H₂₈P₂S₂: C, 73.70; H, 4.81; P, 10.56; s, 10.93 found: C, 73.82; H, 4.52; P, 9.59; S, 10.71. ¹H NMR (CDCl₃, 295 K) δ: 7.80-7.72 (m, 12H), 7.63 (d, 4H), 7.52 (t, 4H), 7.45 (t, 8H). ¹³C NMR (CDCl₃, 295 K) δ: 143.28, 133.50, 133.39, 133.27, 132.80, 132.68, 132.59, 132.05, 128.96, 127.73. ³¹P NMR (CDCl₃, 295 K): δ 43.66.

Treatment of P1 with elemental sulfur even for extended time periods did not afford complete conversion to PS1. TLC indicated the presence of phosphine impurities. Notably, following digestion in methanol impurities were no longer detectable by ³¹P NMR and TLC, yet, impurities were separated from the lower temperature fractions (150-170° C., base pressure 10⁻⁶ Torr for a period of 24 hours) following further purification by high vacuum, gradient temperature sublimation. Threesublimations were performed prior to photophysical and device studies in order to ensure removal of these impurities.

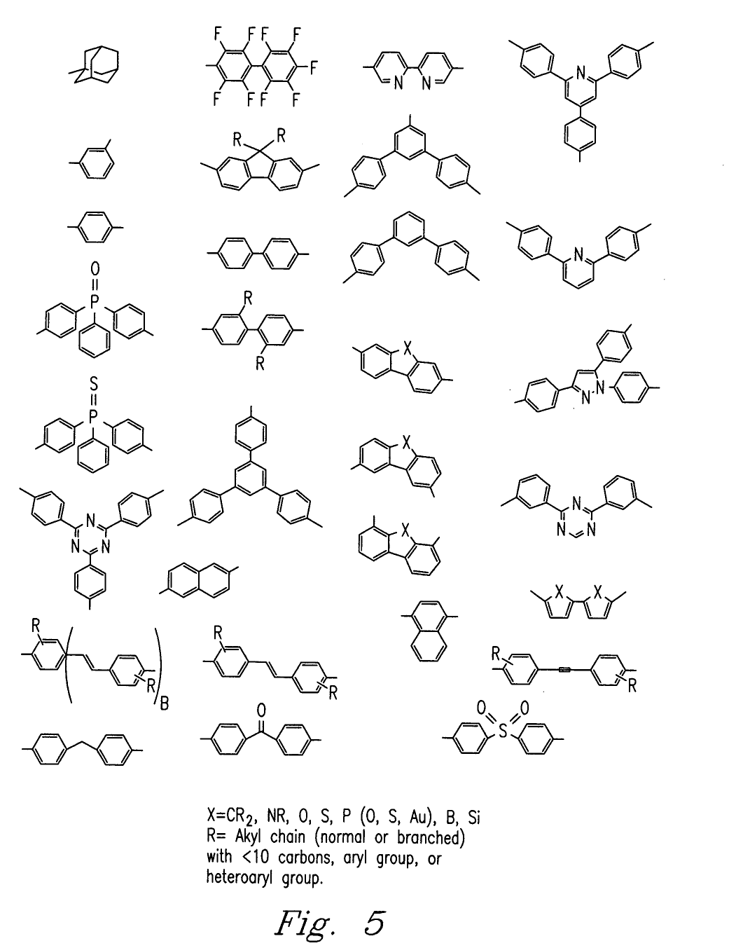

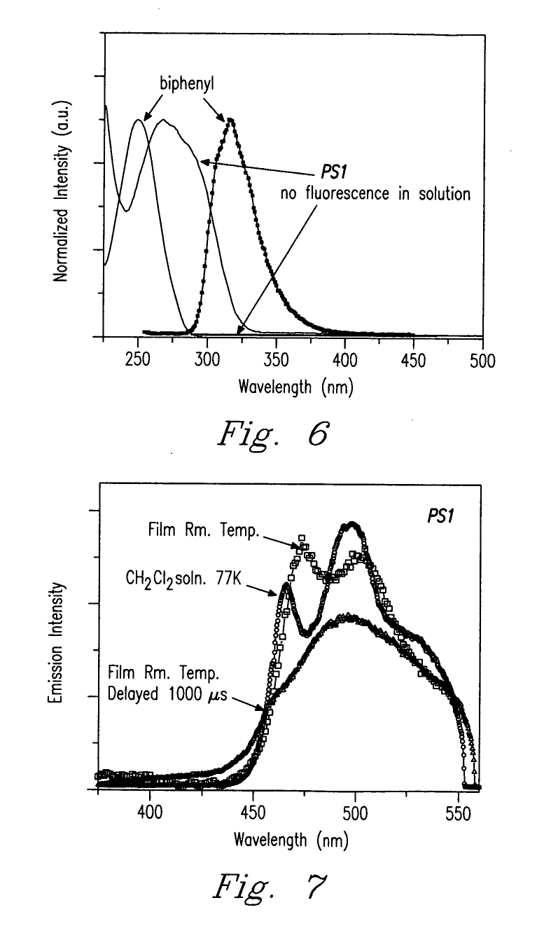

The absorption and luminescence spectra of PS1 and the organic bridging group, biphenyl are presented in FIG. 6. The absorption maximum is 268 nm in solution [CH₂Cl₂, ε×10⁴=3.23], which is shifted by only 18 nm from the organic bridge, biphenyl. PS1 in degassed methylene chloride was excited with a xenon lamp at 280 nm at room temperature, and was shown to be nonemissive in solution, even though the bridging group, biphenyl is emissive in solution as shown in FIG. 6. PS1 in degassed methylene chloride was excited with a xenon lamp at 280 nm at 77 K, and was shown to be emissive in frozen solution, as shown in FIG. 7. Solid state films and crystalline samples of PS1 were also excited with a xenon lamp at 280 nm at room temperature, and were shown to emit blue light at 473 nm, also as shown in FIG. 7. The radiative lifetime was measured in frozen methylene chloride at 77 K using time-resolved fluorimetry (detection delayed by 0.001 s) and was 0.48±0.04 s for the blue emission consistent with phosphorescence, as is also shown in FIG. 7. The phosphorescence energy of PS1 is similar to the phoshorescence energy reported for the bridging group, biphenyl at 77 K, as reported in the literature [see for example, Taylor, et al, J. Am. Chem. Soc. (1973), 95, 3215].

Absorbance spectra were recorded with a Shimadzu UV-2501PC Ultraviolet-Visible (UV-Vis) dual-beam spectrometer. Room temperature emission spectra and triplet lifetimes at 77 K were recorded using a Jobin-Yvon SPEX Fluorolog 2 (450-W Xe lamp) at an excitation wavelength of 280 nm. All solution photophysical studies were conducted on dilute samples (optical density˜0.1-0.2) to prevent self-absorption. Phosphorescence spectra were obtained in CH₂Cl₂ at 77 K at an excitation wavelength of 280 nm and time delay of 300 μs using a nanosecond optical parametric oscillator/amplifier operating at a 10 Hz repetition rate. The output was directed onto the sample and emission was collected at right angles to the excitation and focused into a ⅛ m monochromator with a gated intensified CCD camera to record the spectra. The gate of the CCD camera could be set to reject scattered laser light and short-lived luminescence, allowing the observation of long-lived luminescence. Film samples were prepared on fused quartz by resistive heating from tantalum boats at˜10⁻⁷ torr.

The procedure for preparing an OLED is as follows. On a commercially available indium tin oxide substrate, a simple bilayer electroluminescent device was grown by vacuum evaporation consisting of, in sequence, a 200 Å thick layer of copper phthalocyanine (CuPc), a 400 Å thick layer of PS1 and a cathode consisting of a 6 Å thick LiF layer followed by a 1000 Å thick Al layer. The cathode was deposited through a stencil mask to yield circular devices 1 mm in diameter. A quartz crystal oscillator placed near the substrate was used to measure the thickness of the films, which were calibrated ex situ using ellipsometry. Devices were tested in air with an electrical pressure contact made by means of a 25 μm diameter Au wire. Current-voltage characteristics were measured with an Agilent Technologies 4155B semiconductor parameter analyzer and EL spectra were recorded with an EG&G optical multichannel analyzer on a 0.25 focal length spectrograph.

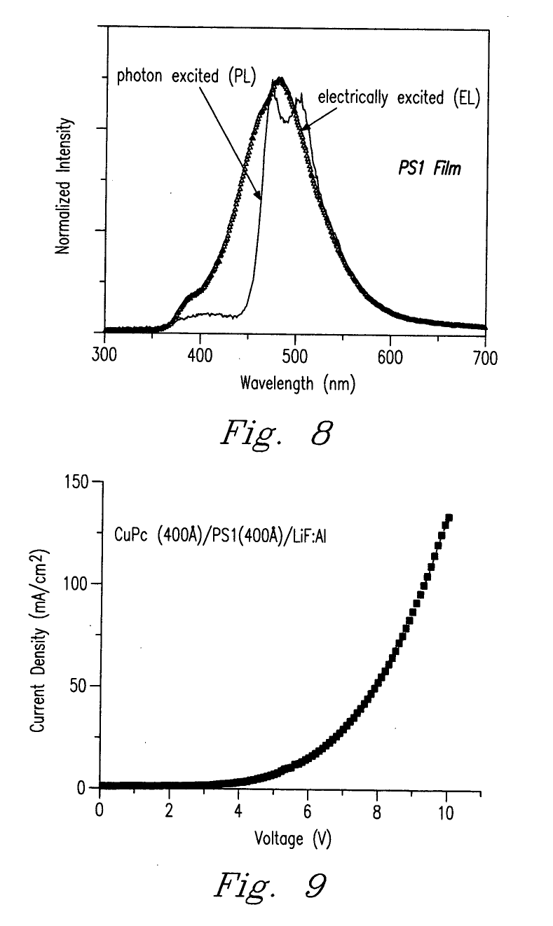

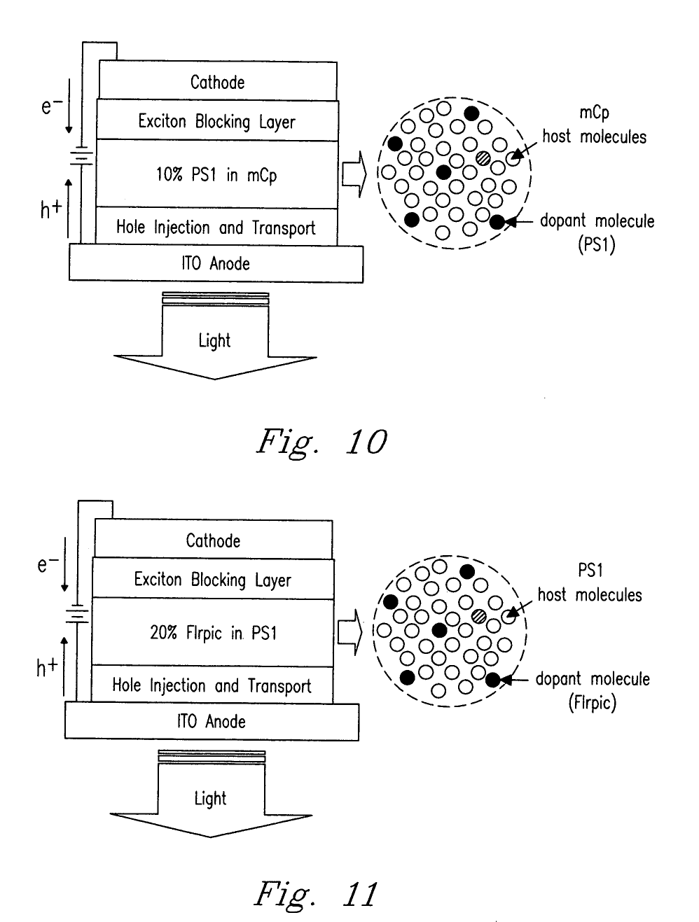

The electroluminescence (EL) spectrum of a simple bilayer OLED grown by vacuum evaporation on indium tin oxide coated glass using PS1 as the active emissive layer is shown in FIG. 8 and is identical to the phosphorescence emission observed from a solid state film at room temperature. A graph of the measured current density verses voltage is shown as FIG. 9. The measured electrophosphorescence efficiency for this device at 13 mA/cm² was 0.1% at 5.8 V. PS1 was also tested as a phosphorescent dopant in a phosphorescent doped OLED configuration as shown in FIG. 10. PS1 was doped into a N,N′-dicarbazolyl-3,5-benzene (mCp) at 10% by weight. The measured electrophosphorescence efficiency for this device at 13 mA/cm² was 0.14% at 8.1 V. Although, these efficiencies are low compared to up to 10% for the state of the art organometallic phosphor dopants, the device structure is non-optimized to use the materials as a phosphorescent dopant.

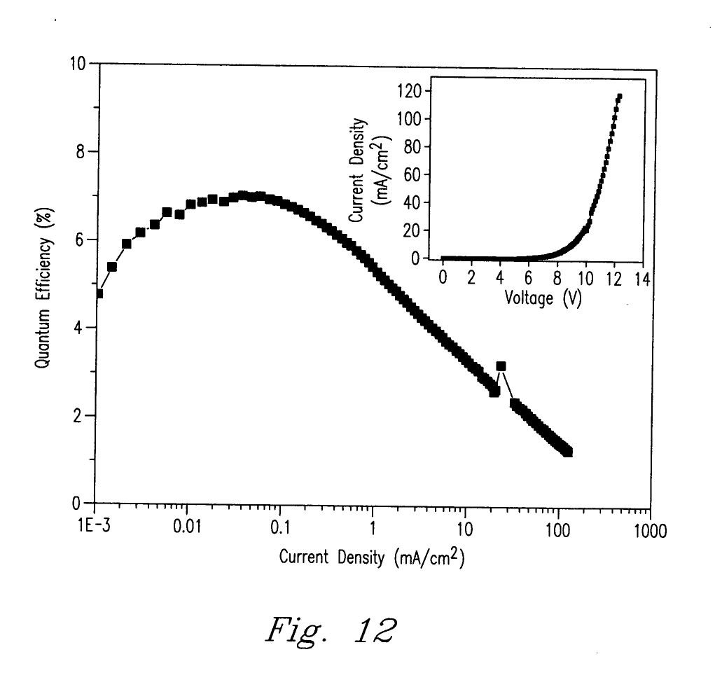

PS1 was tested as a charge transporting host material doped with the blue/green organometallic phosphor, iridium(III)bis(4,6-(di-fluorophenyl)-pyridinato-N,C2.)picolinate (FIrpic). The general device structure is shown in FIG. 11. Hole injection is facilitated by addition of a 50 Å layer of 4,4′,4″-tris(carbazol-9-yl)-triphenylamine (TCTA, purchased from H.W. Sands Corp.) and the hole blocking layer is comprised of 200 Å of PS1. As shown in FIG. 12, external quantum efficiencies approaching 7% are achieved in non-optimized devices. The measured electrophosphorescence efficiency for this device at 13 mA/cm² was 4.5% at 8.5 V. When PS1 is used as the host for FIrpic and PO1 is used as the hole blocking layer, the measured electrophosphorescence efficiency increases at 13 mA/cm² to 6.9% and the voltage lowered to 6.1 V. This efficiency is an improvement over the efficiency shown in the device using 4,4′-bis(diphenylphosphine oxide) biphenyl (PO1) (Burrows, et al, Appl. Phys. Lett. (2006) 88, 1, in press)

Phosphine sulfide materials can also serve as exciton and/or hole blocking layers in green OLEDs. Following again the general device structure of FIG. 11, two identical light emitting structures were made using a well studied green phosphor, bis(2-phenylpyridine) Ir (III) acetylacetonate [Ir(ppy)₂acac] doped into dicarbazoylbiphenyl (CBP). Both PS1 and bathocuproine (BCP), a well known hole and exciton blocking material were each separately used as exciton/hole blocking layers. Both structures were then coated with aluminum tris(8-hydroxyquinoline) (ALQ) as the electron injection layer. The measured electrophosphorescence efficiency for these devices at 13 mA/cm² were 4.7% and 4.1% for PS1 and BCP, respectively, showing that a PS1 exciton/hole blocking layer gives higher efficiencies.

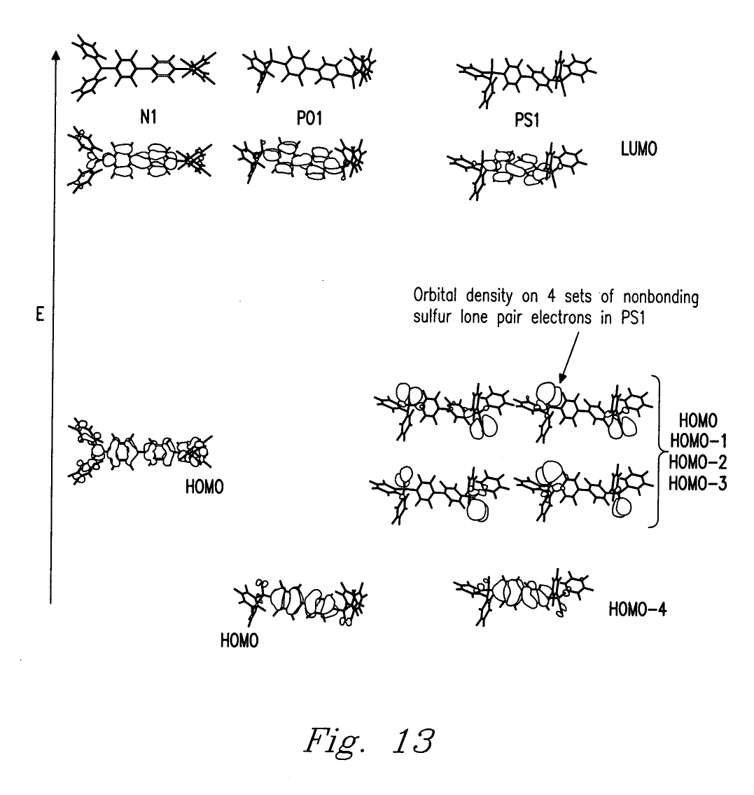

The difference in properties between PS1, PO1 and N1 can be understood by examining the geometries and electronic structures of these materials in terms of bridging group (biphenyl) and outer group (phenyl) domains separated by P═S, P═O or N moieties. The computed structures for PS1, PO1 and N1 are shown in FIG. 13. The N centers are trigonal planar allowing interaction of the nitrogen electron lone pairs with the bridging group and outer groups. In contrast, the distorted tetrahedral geometry and absence of available lone pair electrons on the phosphorus site in both P═S and P═O prevents electron delocalization between the bridging group and outer groups.

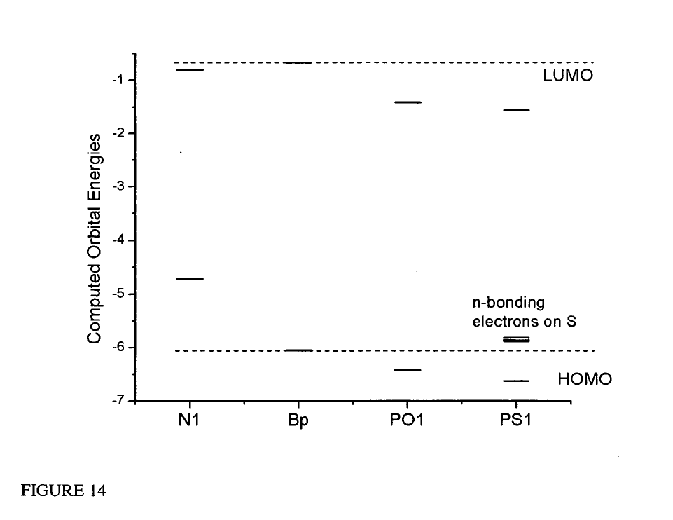

The large blue shift in absorption and emission energies of PS1 and PO1 compared to N1 can be qualitatively attributed to a significant deepening of the occupied manifold (HOMO energy is lowered by˜1.7 eV) and slight lowering of the virtual manifold (LUMO energy is lowered by˜0.6 eV) resulting in a widening of the optical gap by>1 eV. The changes in computed energies of the HOMO and LUMO of the phosphine sulfide (PS1) is compared to the phosphine oxide (PO1), the amine (N1), and all are compared to the bridging group, biphenyl (Bp) as shown in FIG. 14.

For phosphine sulfides, the nature of the P═S bond is different than the P═O bond. Sulfur is a more polarizable element than oxygen, and both functionalities inductively polarize the pi cloud of aromatic substituents which can enhance electron transport. The nonbonding electrons on sulfur dominate the first four degenerate HOMO states when the bridging groups and outer groups are high energy organic chromophores as shown in FIGS. 13 and 14. Hence, the nonbonding electrons are sufficiently coupled to the electronic states of the bridging group, which enhances intersystem crossing from the singlet excited state to the triplet excited state, resulting in no observable fluorescence, but observable room temperature phosphorescence in the solid state characteristic of the bridging group.

These results thus provide an example of the present invention used as the active layer in an OLED, and show that the P═S moieties of PS1 restrict conjugation between bridging and outer groups, and provide a comparison to corresponding P═O moieties. While the phosphine sulfides exhibit similar electron transport properties to analogous phosphine oxide structures, in the present invention, the phosphine sulfides outperform the phosphine oxides as host materials and exhibit phosphorescence instead of fluorescence characteristic of the bridging group.

Closure

While a preferred embodiment of the present invention has been shown and described, it will be apparent to those skilled in the art that many changes and modifications may be made without departing from the invention in its broader aspects. The appended claims are therefore intended to cover all such changes and modifications as fall within the true spirit and scope of the invention.

1) A material comprising one or more phosphine sulfide moieties, the phosphorus atom of each of said phosphine moieties further bonded by single bonds to at least two outer groups, said material substantially purified and configured as part of a circuit.

2) The material of claim 1 further comprising two or more phosphine sulfide moieties joined by one or more bridging groups, wherein the phosphorus atom of at least two of said phosphine sulfide moieties is further bonded by single bonds to two outer groups, said material substantially purified and configured as part of a circuit.

3) The material of claim 2 wherein said bridging groups are selected from the group consisting of aryl, heteroaryl, cycloalkyl, and alkyl groups and substituted derivatives where the substituted group is an alkyl, aryl, heteroaryl, halo, amino, hydroxyl, alkoxy, cyano, halogenated alkyl, aryl or heteroaryl group, and combinations thereof.

4) The material of claim 3 wherein said bridging groups are selected as difunctional or multifunctional groups bonded to said phosphorus atoms at two or more positions and selected from benzene, naphthalene, pyrene, stilbene, diphenylethyne, pyridine, quinoline, thiophene, phenylene vinylene, thienylene vinylene, biphenyl, diphenylmethane, bithiophene, bipyridine, porphyrins and metalloporphyrins, phthalocyanines and metallophthalocyanines, perylene and naphthalene, carbazole, dibenzothiophene, dibenzofuran, 2,4-diphenyl-1,3,5-triazine, 2,4,6- triphenyl-1,3,5-triazine, 2,4,6-triphenylpyridine, diphenylmethane, benzophenone, dibenzosulfoxide.

5) The material of claim 2 wherein said outer groups are selected from the group consisting of aryl, heteroaryl, cycloalkyl, and alkyl groups and substituted derivatives where the substituted group is an alkyl, aryl, heteroaryl, halo, amino, hydroxyl, alkoxy, cyano, halogenated alkyl, aryl or heteroaryl group, and combinations thereof.

6) The material of claim 5 wherein said outer groups are selected from benzene, naphthalene, pyrene, stilbene, diphenylethyne, pyridine, quinoline, thiophene, phenylene vinylene, thienylene vinylene, biphenyl, diphenylmethane, bithiophene, bipyridine, porphyrins and metalloporphyrins, phthalocyanines and metallophthalocyanines, perylene and naphthalene, carbazole, dibenzothiophene, dibenzofuran, 2,4-diphenyl-1,3,5-triazine, 2,4,6- triphenyl-1,3,5-triazine, 2,4,6-triphenylpyridine, diphenylmethane, benzophenone, dibenzosulfoxide.

7) The material of claim 1 and 2 wherein said outer groups are identical.

8) An organic light emitting device having an anode layer, a cathode layer, and at least one organic layer interposed between the anode and cathode layer, wherein at least one of said organic layers comprises a substantially purified material having one or more phosphine sulfide moieties, wherein the phosphorus atoms of each of said phosphine sulfide moieties is further bonded by single bonds to two outer groups.

9) The organic light emitting device of claim 8 wherein said substantially purified material is a charge transport material.

10) The organic light emitting device of claim 9 wherein said charge transport material emits light in response to an external stimulus.

11) The organic light emitting device of claim 9 wherein said charge transport material further contains at least one dopant.

12) The organic light emitting device of claim 11 wherein said charge transport material works in conjunction with said at least one dopant to emit light in response to an external stimulus.

13) The organic light emitting device of claim 11 where the light is emitted substantially from the dopant.

14) The organic light emitting device of claim 8 wherein said substantially purified material functions as a charge blocking material.

15) The organic light emitting device of claim 8 wherein said substantially purified material functions as an exciton blocking material.

16) The organic light emitting device of claim 8 wherein said substantially purified material is a dopant in one of said organic layers and emits phosphorescence in response to an external stimulus at room temperature.

17) The material of claim 1 and 2 wherein the circuit is a photodetector.

18) The material of claim 1 and 2 wherein the circuit is a solar cell.

19) The material of claim 1 and 2 wherein the circuit is a thin film transistor.

20) The material of claim 1 and 2 wherein the circuit is a bipolar transistor.

21) The material of claim 1 and 2 wherein the circuit is incorporated in an array to form an information display.
